(19) United States
(12) Patent Application Publication
(10) Pub. No.: US 2016/0073993 A1
(43) Pub. Date: Mar. 17, 2016

(54) SYSTEM AND METHOD FOR CORRECTING PET IMAGING DATA FOR MOTION USING MR IMAGING DATA AND TRACKING COILS

(71) Applicants: Jinsong OUYANG, Lexington, MA (US); Jerome L ACKERMAN, Newton, MA (US); Chuan HUANG, Boston, MA (US); Yoann PETIBON, Cambridge, MA (US); Georges ELFAKHRI, Brookline, MA (US)

(72) Inventors: Jinsong Ouyang, Lexington, MA (US); Jerome L. Ackerman, Newton, MA (US); Chuan Huang, Boston, MA (US); Yoann Petibon, Cambridge, MA (US); Georges El Fakhri, Brookline, MA (US)

(21) Appl. No.: 14/787,292

(22) PCT Filed: Apr. 30, 2014
(86) PCT No.: PCT/US14/36132

§ 371 Date: Oct. 27, 2015

Related U.S. Application Data
(60) Provisional application No. 61/817,750, filed on Apr. 30, 2013.

(51) Int. Cl. A61B 6/00; G01R 33/48; A61B 6/03

(52) U.S. Cl. CPC A61B 6/527; A61B 6/037; G01R 33/481

(57) ABSTRACT

A system and method for generating motion-corrected positron emission tomography (PET) images is provided. A subject is prepared to be imaged by arranging a plurality of motion tracking coils about the subject. Each of the plurality of motion tracking coil includes a conductor extending through a plurality of loops to form a coil and includes a sample material. PET coincidence events are acquired concurrently with magnetic resonance (MR) data generated using the plurality of motion tracking coils with an MR system. Motion data is determined from the MR data and is incorporated in PET reconstruction or applied to PET images to generate motion corrected PET images.

### CROSS-REFERENCE TO RELATED APPLICATIONS

This application is based on, claims priority to, and incorporates herein by reference U.S. Provisional Application Ser. No. 61/817,750, filed Apr. 30, 2013, and entitled, “Motion Tracking Micro-Coils.”

### BACKGROUND OF THE INVENTION

The present disclosure relates generally to medical imaging and, more particularly, to systems and methods for tracking motion during medical imaging procedures using motion tracking coils.

Positrons are positively charged electrons which are emitted by radionuclides that have been prepared using a cyclotron or other device. These are employed as radioactive tracers called “radiopharmaceuticals” by incorporating them into substances, such as glucose or carbon dioxide. The radiopharmaceuticals are administered to a patient and become involved in biochemical or physiological processes such as blood flow; fatty acid and glucose metabolism; and protein synthesis.

As the radionuclides decay, they emit positrons. The positrons travel a very short distance before they encounter an electron, and when this occurs, they are annihilated and converted into two photons, or gamma rays. This annihilation event is characterized by two features which are pertinent to PET scanners—each gamma ray has an energy of 511 keV and the two gamma rays are directed in nearly opposite directions. An image indicative of the tissue concentration of the positron emitting radionuclide is created by determining the number of such annihilation events at each location within the field of view.

A conventional positron emission tomography (PET) imaging system includes one or more rings of detectors which encircle the patient and which convert the energy of each 511 keV photon into a flash of light that is sensed by a photomultiplier tube (PMT). Coincidence detection circuits connect to the detectors and record only those photons which are detected simultaneously by two detectors located on opposite sides of the patient. The number of such simultaneous events indicates the number of positron annihilations that occurred along a line joining the two opposing detectors. Within a few minutes hundreds of million of events are recorded to indicate the number of annihilations along lines joining pairs of detectors in the ring. These numbers are employed to reconstruct an image using well known computed tomography techniques.

Many minutes are typically required to accumulate a sufficient number of counts in a PET imaging system in order to reconstruct an image having sufficient SNR to be of clinical value. During that time period the subject of the examination is prone to move at least one or more times. As a result, the image that is reconstructed is often blurred.

For example, PET scans are an important part in the diagnosis, prognostication, and monitoring of dementia, which represents a patient group that is particularly susceptible to motion. That is, artifacts from head motion are the major challenge facing brain PET. This is particularly true in the elderly, with increased significance in patients with dementia or movement disorders. Also, voluntary patient body (other than brain) motion is also common in clinical body PET imaging (studies shown 29% prevalence in dynamic myocardial perfusion PET), which degrades the quality of the PET images and affect the diagnostic value of the PET exam, possibly leading to misdiagnosis.

Many approaches have been explored in the effort to correct motion artifacts. Depending on whether the motion is estimated from the acquired PET data or by other instrumentation, the approaches can be divided into two groups: auto-correction and assisted-correction. For the auto-correction techniques, the measured PET data are divided into temporal frames, and the motion is then estimated between temporal frames from the PET data. The estimated motion field can then be used to transform the reconstructed images (Friston et al., 1995; Tellmann et al., 2006; Woods et al., 1992) or the sinograms (Hutton et al., 2002; Kyme et al., 2003) of each temporal frame to a reference frame. The accuracy of motion estimation using this approach is limited by the noise of PET images, which increases as the data set is divided into temporal frames for a dynamic image sequence. Moreover, the fact that the motion estimation relies on the generation of images or sinograms limits its temporal resolution. Thus, such methods are not suitable when the activity distribution is fast changing or the object is fast moving. The reconstruction algorithms of the assisted-correction approaches are similar to auto-correction techniques except that the motion information is instead measured using an instrument other than the PET camera, such as video/infrared cameras (Bloomfield et al., 2003; Goldstein et al., 1997; Picard and Thompson, 1997), and approaches with structured light (Olesen et al., 2012, 2013).

Similar approaches have also been applied to motion correction in MRI (Schulz et al., 2012; Zaitsev et al., 2006). One advantage of the optical motion tracking approaches is that they are independent of the MRI acquisition, so that no changes to the MR pulse sequence are required, in contrast to MR navigator-based methods. Another advantage is that the optical methods, in principle, are capable of achieving high frame rate. Some of these approaches monitor the motions of reflectors attached to the subject's head and some observe a portion of the subject's face. In every case, these methods require an unobstructed view from the cameras to the reflectors or the subject's face. This is challenging for combined PET-MR systems performing head studies because the view from outside of the scanner is blocked by the MR head coil, especially for modern head coils with a large number of channels. There are RF contamination and MR compatibility issues associated with installing cameras inside of the scanner. Moreover, these optical systems require complicated calibrations.

Conventional MR navigator methods (Ehman and Felmlee, 1989; Wang et al., 1996) can be used to track motion with temporal resolution less than 20 ms. However, such methods cannot be used to track head rotation. Catana et al. (2011) used the cloverleaf navigator method (van der Kouwe et al., 2006) to track head motion for PET motion compensation. Although this method can track both translation and rotation, its motion tracking accuracy suffers from off-resonance effects, gradient instabilities, as well as signal contamination from non-rigid motion of the neck. Moreover, such cloverleaf-navigator methods require approximately 20 s of motionless data to calibrate. Petibon et al. (2013) used image-based MR motion tracking for non-rigid motion compensation in cardiac PET. However, it generally lacks temporal resolution because of the long scanning time needed for acquiring the entire image volume.

Therefore, there is a need for systems and methods to control or offset motion, particularly, in PET imaging.

### SUMMARY OF THE INVENTION

The present disclosure provides system and methods that overcome the aforementioned and other drawbacks. Particularly, a plurality of coils, tracked by magnetic resonance imaging (MRI), are used to determine head or body motion in real-time during a PET acquisition and incorporate the measured motion in the PET imaging reconstruction process or apply the measured motion to the corresponding PET image reconstructed at each motion frame. Each tracking coil can be wound around a sealed doped water sample to receive only the signal from the sample. A projection of the sample contains a single sharp peak indicating the location of the tracking coil along the field gradient. Thus, three orthogonal projections can be used to yield the tracking coil position in 3D space, which can be performed at high speed. The temporal resolution for tracking coil tracking can be better than 15 ms. The motion measured by MR tracking coils can then be used for motion correction in the PET image reconstruction process.

In accordance with one aspect of the disclosure, a medical imaging system is disclosed that includes a positron emission tomography (PET) system for acquiring a series of medical images of a subject, the PET system. The PET system includes a plurality of detectors arranged about a bore configured to receive the subject and to acquire gamma rays emitted from the subject as a result of a radiotracer administered to the subject and configured to communicate PET signals corresponding to acquired gamma rays. The medical imaging system also includes a magnetic resonance imaging (MRI) system for acquiring a series of medical images of the subject from within the bore. The MRI system includes a magnet system configured to generate a polarizing magnetic field about at least a portion of the subject arranged in the bore and a plurality of gradient coils configured to apply a gradient field to the polarizing magnetic field. The MRI system also includes a radio frequency (RF) system having a plurality of motion tracking coils positioned, the RF system configured to acquire MR data from the motion tracking coils, wherein each motion tracking coil includes a conductor extending through a plurality of loops to form a coil. The MRI system further includes an MRI computer system programmed to control the RF system to receive MR data from the RF system. The medical imaging system also includes a data processing system configured to receive the MR data and determine motion of the subject from the MR data by determining a position of the motion tracking coils over time and receive the PET signals and correct the PET signals using the motion of the subject determined from the MR data to generate corrected PET images of the subject.

In accordance with another aspect of the disclosure, a method is disclosed for generating a positron emission tomography (PET) image corrected for subject motion in a combination PET and magnetic resonance imaging (MRI) system. The method includes preparing a subject to be imaged in a combined PET and MR imaging process by administering a radionuclide to the subject and arranging a plurality of motion tracking coils about the subject, wherein each of the plurality of motion tracking coil includes a conductor extending through a plurality of loops to form a coil configured to receive signals from a sample material. The method also includes acquiring, with a PET imaging system, sinogram data that counts a number of coincidence events at a plurality of lines of response (LOR) and periodically acquiring, with the MRI system, over a time period that extends concurrently with acquiring the sinogram data, a plurality of MR signals generated using the plurality of motion tracking coils arranged about the subject. The method also includes calculating, from the MR data, motion data and correcting the sinogram data using the motion data to generate corrected PET images of the subject.

The foregoing and other aspects and advantages of the disclosure will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a pictorial view of a cross-section of a combination positron emission tomography (PET) imaging system and magnetic resonance imaging (MRI) system which employs the present invention.

FIG. 2 is a schematic diagram of the PET imaging system portion of the system of FIG. 1.

FIG. 3 is a schematic diagram of the MRI system portion of the system of FIG. 1.

FIG. 4 is a pictorial representation of a tracking coil system in accordance with the present disclosure.

FIG. 5A is a pulse sequence diagram illustrating a pulse sequence for tracking motion.

FIG. 5B is a pulse sequence diagram illustrating another pulse sequence for tracking motion.

FIG. 5C is a schematic diagram illustrating a k-space sampling strategy in accordance with the present disclosure.

FIG. 6 is a flow chart setting forth the steps of a process for correcting PET image data using MR data to track motion during the acquisition of the PET data.

FIG. 7 is a schematic diagram illustrating the effect of motion on PET data.

FIG. 8 is a graph of projections of a tracking coil containing a sample material in accordance with the present disclosure attached to a cylindrical water phantom with varying strength of the spoiler gradient.

FIG. 9 is a graph showing the effect of magnetic susceptibility on position readout.

### DESCRIPTION OF THE PREFERRED EMBODIMENT

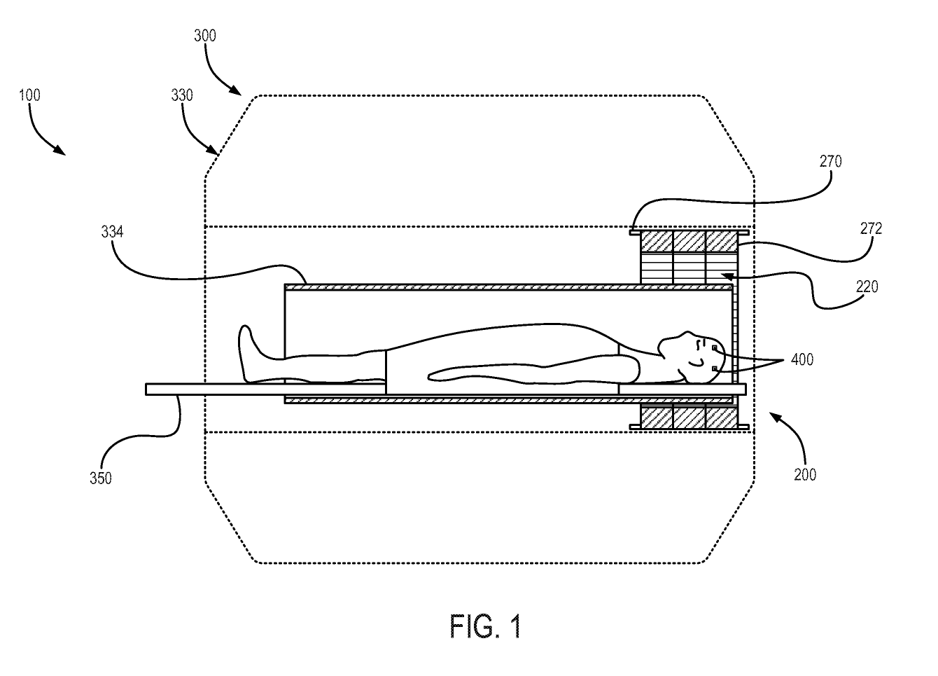

Referring to FIG. 1, the present disclosure may be implemented using a combined or simultaneous MR/PET system 100. The system 100 can be conceptualized as including an MRI system 300 having a cylindrical magnet assembly 330 which receives a subject to be imaged. Disposed within the magnet assembly 330 is a PET system 200 that includes plurality of PET detector rings 272 supported by a cylindrical PET gantry 270. Accordingly, each detector ring 272 has an outer diameter dimensioned to be received within the geometry of the MRI system 300. In other configurations, a single PET detector ring may be utilized. A patient table 350 is provided to receive a patient to be imaged. The gantry 270 is slidably mounted on the patient table 350 such that its position can be adjusted within the magnet assembly 330 by sliding it along the patient table 350. An RF coil 334 is employed to acquire MR signal data from a patient and is positioned between the PET detector rings 272 and the patient to be imaged. PET and MR data acquisitions are carried out on the patient, either simultaneously, in an interlaced or interleaved manner, or sequentially. Combined PET/MR imaging systems have been described, for example, in U.S. Pat. No. 7,218,112 and in U.S. Patent Application No. 2007/0102641, which are incorporated herein by reference. As will be described, the present disclosure provides a tracking system 400 that may be mounted to or coupled with a patient to track motion of the patient using the MRI system 300 and correct images reconstructed using data acquired with the PET system for the tracked motion.

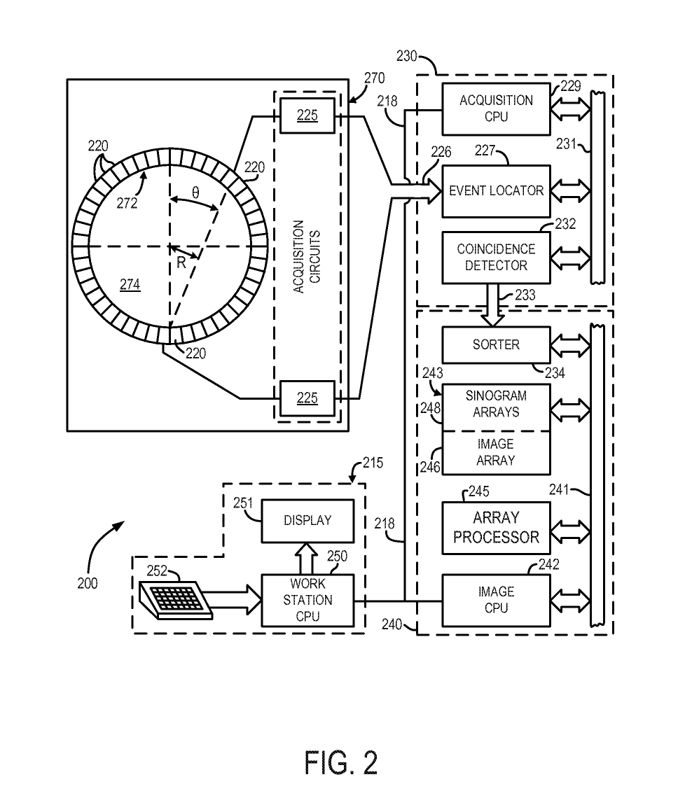

Referring particularly to FIG. 2, the PET system 200 includes the gantry 270, which supports the detector ring assembly 272. The detector ring 272 includes detector units 220. The signals produced by the detector units 220 are then received by a set of acquisition circuits 225, which produce digital signals indicating the line of response and the total energy. These signals are sent through a communications link 226 to an event locator circuit 227. Each acquisition circuit 225 also produces an event detection pulse (EDP) which indicates the exact moment the scintillation event took place.

The event locator circuits 227 form part of a data acquisition processor 230, which periodically samples the signals produced by the acquisition circuits 225. The processor 230 has an acquisition CPU 229 which controls communications on local area network 218 and a backplane bus 231. The event locator circuits 227 assemble the information regarding each valid event into a set of digital numbers that indicate precisely when the event took place and the position of the scintillator crystal which detected the event. This event data packet is conveyed to a coincidence detector 232 which is also part of the data acquisition processor 230.

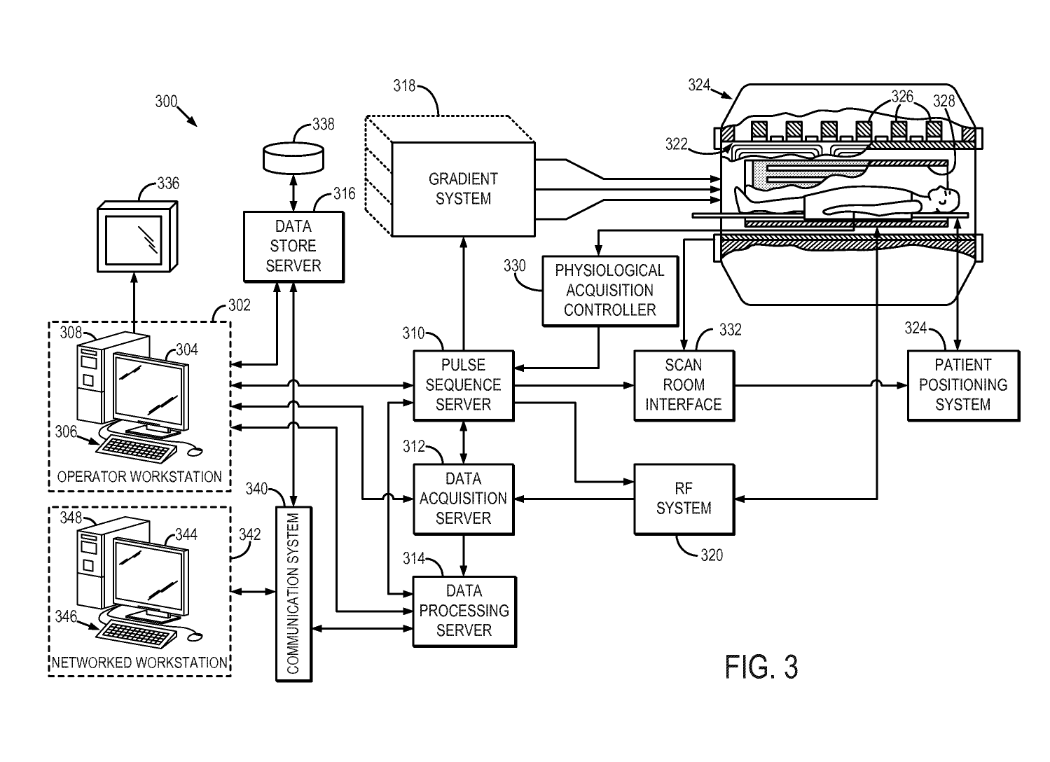

The coincidence detector 232 accepts the event data packets from the event locators 227 and determines if any two of them are in coincidence. Coincidence is determined by a number of factors. First, the time markers in each event data packet must be within a preset time of each other, and second, the locations indicated by the two event data packets must lie on a straight line. Events that cannot be paired are discarded, but coincident event pairs are located and recorded as a coincidence data packet. As will be described, the coincidence data packets can be corrected for motion of the subject during the acquisition using information received from the MRI system 300 of FIGS. 1 and 3. Using this corrective information and the information in each coincidence data packet, a corresponding set of corrected coincidence data packets can be calculated. As will be described, each coincidence data packet can, thus, be corrected to change its projection ray, (R, θ) by an amount corresponding to the movement of the subject, as determined using information from the MRI system 300 of FIGS. 1 and 3.

The corrected coincidence data packets are conveyed through a link 233 to a sorter 234 where they are used to form a sinogram. This corrective process is repeated each time corrective values are received from the MRI system. The correction is made on those coincidence data packets that have accumulated since the receipt of the previous corrective values.

The sorter 234 forms part of an image reconstruction processor 240. The sorter 234 counts all events occurring along each projection ray (R, θ) and organizes them into a two dimensional sinogram array 248 which is stored in a memory module 243. In other words, a count at sinogram location (R, θ) is increased each time a corrected coincidence data packet at that projection ray is received. Due to the corrections made to the coincidence events, the sinogram that is formed during the scan depicts the subject being examined in the reference position despite subject motion that occurs during the scan. The image reconstruction processor 240 also includes an image CPU 242 that controls a backplane bus 241 and links it to the local area network 218. An array processor 245 also connects to the backplane 241 and it reconstructs an image from the sinogram array 248. The resulting image array 246 is stored in memory module 243 and is output by the image CPU 242 to the operator work station 215.

The operator work station 215 includes a CPU 250, a display 251 and a keyboard 252. The CPU 250 connects to the network 218 and it scans the keyboard 252 for input information. Through the keyboard 252 and associated control panel switches, the operator can control the calibration of the PET scanner and its configuration. Similarly, the operator can control the display of the resulting image on the display 251 and perform image enhancement functions using programs executed by the work station CPU 250.

Referring to FIG. 3, the MRI system 300 is illustrated in further detail. The MRI system 300 includes an operator workstation 302, which will typically include a display 304; one or more input devices 306, such as a keyboard and mouse; and a processor 308. The processor 308 may include a commercially available programmable machine running a commercially available operating system. The operator workstation 302 provides the operator interface that enables scan prescriptions to be entered into the MRI system 300. In general, the operator workstation 302 may be coupled to four servers: a pulse sequence server 310; a data acquisition server 312; a data processing server 314; and a data store server 316. The operator workstation 302 and each server 310, 312, 314, and 316 are connected to communicate with each other. For example, the servers 310, 312, 314, and 316 may be connected via a communication system 340, which may include any suitable network connection, whether wired, wireless, or a combination of both. As an example, the communication system 340 may include both proprietary or dedicated networks, as well as open networks, such as the internet.

The pulse sequence server 310 functions in response to instructions downloaded from the operator workstation 302 to operate a gradient system 318 and a radiofrequency (RF) system 320. Gradient waveforms necessary to perform the prescribed scan are produced and applied to the gradient system 318, which excites gradient coils in an assembly 322 to produce the magnetic field gradients Gₓ, G_{y}, and G_{z} used for position encoding magnetic resonance signals. The gradient coil assembly 322 forms part of a magnet assembly 324 that includes a polarizing magnet 326 and optionally a whole-body RF coil 328.

RF waveforms are applied by the RF system 320 to the RF coil 328, or a separate local coil, such as the tracking coil system 400, as illustrated in FIG. 1, in order to perform the prescribed magnetic resonance pulse sequence. Responsive magnetic resonance signals detected by the RF coil 328, or separate local or tracking coil(s) 400, are received by the RF system 320, where they are amplified, demodulated, filtered, and digitized under direction of commands produced by the pulse sequence server 310. The RF system 320 includes an RF transmitter for producing a wide variety of RF pulses used in MRI pulse sequences. The RF transmitter is responsive to the scan prescription and direction from the pulse sequence server 310 to produce RF pulses of the desired frequency, phase, and pulse amplitude waveform. The generated RF pulses may be applied to the whole-body RF coil 328 or to one or more local coils or coil array 400.

The RF system 320 also includes one or more RF receiver channels. Each RF receiver channel includes an RF preamplifier that amplifies the magnetic resonance signal received by the coil 328 or by the local coil or optionally by a coil in the coil array 400 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components M=\sqrt{I²+Q²} and the phase of the received magnetic resonance signal may also be determined according to the following relationship

$$ϕ = {tan}^{- 1} ( \frac{Q}{I} ) .$$

The pulse sequence server 310 also optionally receives patient data from a physiological acquisition controller 330. By way of example, the physiological acquisition controller 330 may receive signals from a number of different sensors connected to the patient, such as electrocardiograph (ECG) signals from electrodes, or respiratory signals from a respiratory bellows or other respiratory monitoring device. Such signals are typically used by the pulse sequence server 310 to synchronize, or “gate,” the performance of the scan with the subject's heart beat or respiration.

The pulse sequence server 310 also connects to a scan room interface circuit 332 that receives signals from various sensors associated with the condition of the patient and the magnet system. It is also through the scan room interface circuit 332 that a patient positioning system 334 receives commands to move the patient to desired positions during the scan.

The digitized magnetic resonance signal samples produced by the RF system 320 are received by the data acquisition server 312. The data acquisition server 312 operates in response to instructions downloaded from the operator workstation 302 to receive the real-time magnetic resonance data and provide buffer storage, such that no data is lost by data overrun. In some scans, the data acquisition server 312 does little more than pass the acquired magnetic resonance data to the data processor server 314. However, in scans that require information derived from acquired magnetic resonance data to control the further performance of the scan, the data acquisition server 312 is programmed to produce such information and convey it to the pulse sequence server 310. For example, during prescans, magnetic resonance data is acquired and used to calibrate the pulse sequence performed by the pulse sequence server 310. As another example, navigator signals may be acquired and used to adjust the operating parameters of the RF system 320 or the gradient system 318, or to control the view order in which k-space is sampled. In still another example, the data acquisition server 312 may also be employed to process magnetic resonance signals used to determine patient motion, as will be described, and communicate such to the PET system 200 described with respect to FIG. 2 to perform motion correction or compensation. By way of example, the data acquisition server 312 acquires magnetic resonance data and processes it in real-time to produce information that is used to control the overall operation of the MR and PET imaging acquisitions.

The data processing server 314 receives magnetic resonance data from the data acquisition server 312 and processes it in accordance with instructions downloaded from the operator workstation 302. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 314 are conveyed back to the operator workstation 302 where they are stored. Real-time images are stored in a data base memory cache (not shown in FIG. 3), from which they may be output to operator display 312 or a display 336 that is located near the magnet assembly 324 for use by attending physicians. Batch mode images or selected real time images are stored in a host database on disc storage 338. When such images have been reconstructed and transferred to storage, the data processing server 314 notifies the data store server 316 on the operator workstation 302. The operator workstation 302 may be used by an operator to archive the images, produce films, or send the images via a network to other facilities.

The MRI system 300 may also include one or more networked workstations 342. By way of example, a networked workstation 342 may include a display 344; one or more input devices 346, such as a keyboard and mouse; and a processor 348. The networked workstation 342 may be located within the same facility as the operator workstation 302, or in a different facility, such as a different healthcare institution or clinic.

The networked workstation 342, whether within the same facility or in a different facility as the operator workstation 302, may gain remote access to the data processing server 314 or data store server 316 via the communication system 340. Accordingly, multiple networked workstations 342 may have access to the data processing server 314 and the data store server 316. In this manner, magnetic resonance data, reconstructed images, or other data may exchanged between the data processing server 314 or the data store server 316 and the networked workstations 342, such that the data or images may be remotely processed by a networked workstation 342. This data may be exchanged in any suitable format, such as in accordance with the transmission control protocol (TCP), the internet protocol (IP), or other known or suitable protocols.

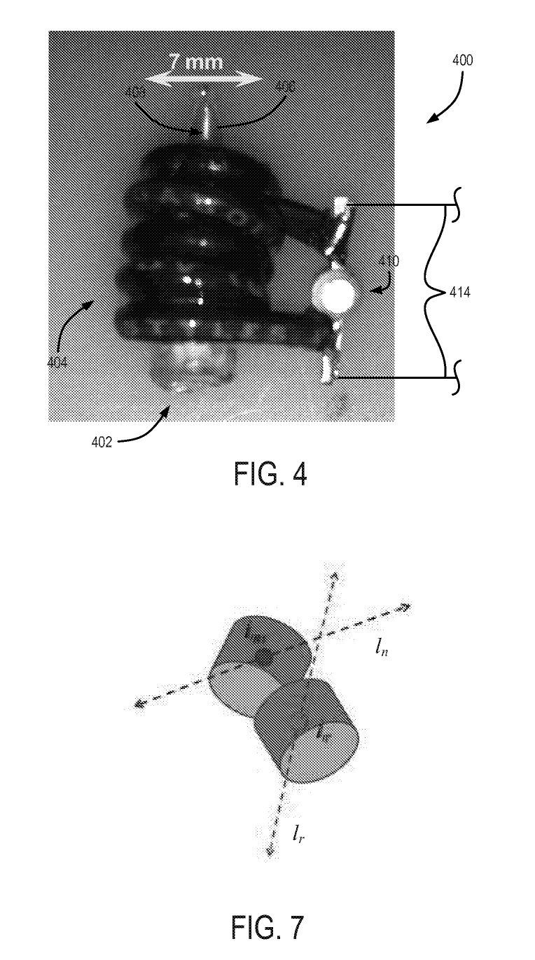

Referring to FIG. 4, each tracking coil in the above-described coil array 400 may serve as an active marker during an MR imaging process, such as using the MR system 300 described above with respect to FIG. 3, performed in coordination with an PET imaging process, such as using the PET system of FIG. 2, so that acquired PET data can be corrected for motion using the acquired MR data. Specifically, one or a plurality of coil arrays 400 may be positioned near or in a region of interest (ROI) of the patient, such as illustrated in FIG. 1 to assist with tracking motion of the subject. The tracking coil or coil systems 400 may be formed of a solenoidal tracking coil 404 defining an interior and an exterior. The tracking coil 404 may extend about a periphery of a sample material 402, as illustrated or may be arranged proximate to a sample material. The sample material 402, as illustrated, may be a water sample that is located in the interior of the tracking coil 404. Alternatively, the sample material may be a portion of the subject arranged proximate to the tracking coil 404. The tracking coil 404, as illustrated, follows a continuous arcuate path to form a cylindrical coil of wire that reflects a solenoid geometry. If the sample material 402 is a water sample, the sample material 402 may be formed as a collection of water molecules arranged inside a housing 406. The housing may be invisible to MR, such as formed of glass or plastic and enclose a water sample 408. In one non-limiting example, the sample material 402 may be formed as a spherical microsample cell with a volume of 18 μL (model 529-A, Wilmad-178 LabGlass, Vineland, N.J., USA). Alternatively, as noted, the tracking coil may detect signals from a sample material adjacent to the exterior of the coil or from a portion of the subject's body adjacent the exterior of the coil as an alternative to or even in addition to utilizing a sample material in its interior.

The water sample 408 inside the housing 406 may be doped to yield short T1 and/or T2 relaxation times. To this end, in the non-limiting example provided above, the sphere can be filled with a degassed solution of deionized water doped with 1.25 g/L NiSO4.6H2O and 5 g/L 180 NaCl. The sphere can be immersed in liquid nitrogen while the air is pumped out, and the neck was flame-sealed under vacuum. The tracking coil 404 may wrap around the housing 406 and be connected to one or more capacitors 410 to resonate the coil at the Lamor frequency of the MR system, such as the MR system 300 of FIG. 3, and to match the electrical impedance of the coil circuit to the impedance of the coaxial cable if the coil circuit is connected to a preamplifier. For example, in one non-limiting example, the MR tracking coils can be built by winding 4 turns of 14 AWG (American wire gauge; 1.63 mm diameter) bare copper wire into a solenoid about 9.1 mm long by 9.8 mm outside diameter, resulting in an inductance of about 82 nH. The capacitance required to resonate the tracking coils at 123.14 MHz (the Larmor frequency of the Siemens 3T scanner) is about 20 pF, which can be created with a combination of a fixed ATC (American Technical Ceramics, Huntington Station, N.Y., USA) non-magnetic chip capacitor and an adjustable Johanson type 9341 190 nonmagnetic surface mount device (SMD) trimmer capacitor (Johanson Manufacturing Corporation, Boonton, N.J., USA).

As illustrated in FIG. 1, a plurality of coil systems 400 may be distributed about a region of interest (in FIG. 1, the subjects head) to track a motion of the region of interest during the imaging process. To this end, the tracking coil system 400 functions as a set of markers to perform location tracking by employing a tracking MR pulse sequence module. The RF excitation of the material to be detected by each coil in the tracking coil system 400 may be accomplished by excitation by the body RF coil 328, by a local RF coil, or by a wired connection, for example by a coaxial cable, to the tracking coil. Alternatively, the RF excitation of the material may be accomplished by the magnetic inductive coupling from the body RF coil 328 or a local RF coil to the tracking coil. To communicate signals received by the coil system 400, as illustrated in FIG. 4, the coil system 400 may utilize the magnetic inductive coupling to the body RF coil 328 or to a local coil of the MRI system 300, the signals thereby being wirelessly transmitted to one or more preamplifiers of the MRI scanner. Alternatively, the signals may be transmitted by wires 414, for example a coaxial cable, to one or more preamplifiers of the MR system 300 of FIG. 3.

If communicating wirelessly using magnetic inductive coupling no impedance matching circuitry is needed. The capacitor 410 may be adjustable to allow the coil system 400 to be fine-tuned with the water sample 402 in place by observing the resonance frequency while the coil system 400 is probed with a coupling loop connected, for example, to a Hewlett-Packard 8753C vector network analyzer displaying reflected power. Alternatively, the capacitor 410 may be a fixed value capacitor, and the coil circuit resonance frequency fine-tuned by adjusting the spacing of the coil windings. In another alternative, the coil circuit resonance frequency may be fine-tuned by adjusting the capacitor and winding spacing in combination.

The typical tracking coil quality factor Q (a dimensionless measure of the sharpness of coil tuning) was tested and, in the above-described non-limiting example, was on the order of 120 with the water sample inserted. The coil system 400 was enclosed in polypropylene tubes to prevent contact with the subject. Additional tracking coils were built similarly with finer gauge, e.g., 26 AWG, insulated magnet wire using a larger number of turns in a close-wound solenoid. The tracking coils using finer wire tended to exhibit higher Q but less stability in tuning, presumably because the finer wire was less rigid, this permitted inductance changes during handling. None of the tracking coils in this study included back-to-back switching diodes to limit the RF excitation applied to the water sample, because the diodes tended to lower the Q as well as the detection sensitivity. In some cases propylene tubes (syringe barrels) were used to support the tracking coil from the inside and provide a “handle” so that the coils could be handled or fixed to the object being imaged.

In operation, the coil system 400 can work by inductive coupling with the coil 328 of the RF system 320 (and with the separate receiver coil if used). If the coil system 400 is not wired to the MRI system 300, but rather communicates wirelessly by inductive coupling, the RF magnetic field of the transmit pulse excites an RF current in the tracking coil 404 of the coil system 400, which in turn excites the spins in the water sample 402. Although the mutual inductance between the coil 328 of the RF system 320 and the tracking coil system 400 is quite small, the RF field (B₁) produced in the water sample 402 is quite large because of the large filling factor and the high quality factor (Q-factor) of the tracking coil 404, such that an RF flip angle of only a fraction of a degree (measured with respect to the body coil for the imaged subject, not the tracking sample) excites an intense signal from the coil system 400. Thus, mutual inductance between the coil system 400 and the coil 328 of the RF system 320 couples the MR signal of the water sample 402 into the receiver, yielding a readily detectable signal.

The location of the coil system 400 can be readily tracked using a variety of MR pulse sequences. However, as will be described, one particular pulse sequence includes 3-6 short-duration RF excitations followed by a gradient-echo readout that yields the projections of the markers onto certain axes. The projections (often X, Y and Z projections) are then combined to generate the 3D location of the marker in the MRI system.

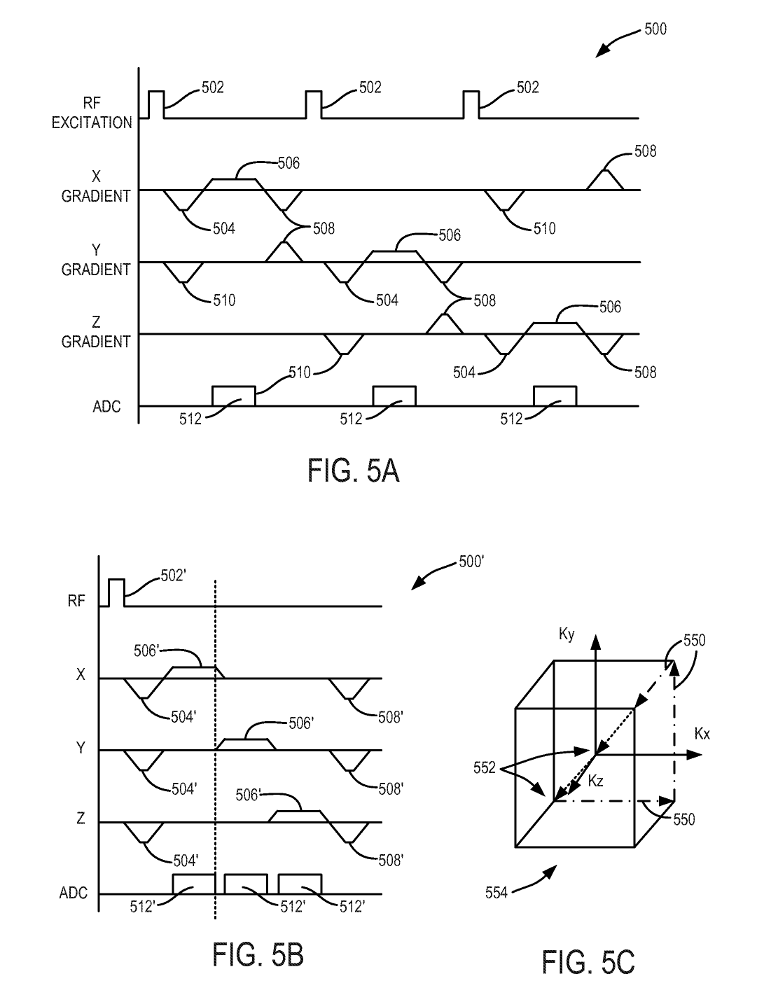

Referring to FIG. 5A, tracking sequence module 500 is illustrated. The tracking sequence module 500 may include separate RF excitations 502 and corresponding pre-positioning 504, readout 506, and rewinding gradients 508 for each of the X, Y and Z projections. Also, spoiling gradient pulses (spoilers) 510 may be used to remove signal contamination from the background object. The spoiler dephases magnetization in objects with substantial thickness along the gradient direction (for example, a subject's head), but has relatively less effect on thin objects. This preserves signal from the very small tracking samples 402 relative to thick objects. A judicious choice of amplitude for the spoiler gradients 510 permits the signals from the subject to be largely suppressed, while retaining the tracking signal 512. For example, a gradient moment inducing a phase dispersion of 2π over an object of thickness 10 cm (largely suppressing its signal) only produces an insignificant phase dispersion of 0.06π over the 3 mm thickness of the tracking sample resulting in essentially no suppression of the tracking signal.

Although intuitively, it might be thought that the trajectory to acquire the volume projections needs to go through the center of the k-space, the application of the spoiler gradient 510 effectively translates the k-space position in the direction perpendicular to the tracking readout direction.

In the tracking pulse sequence module 500 illustrated in FIG. 5A, a significant portion of the time is used for magnetization preparation instead of data acquisition. The efficiency of the sequence, defined as the time spent sampling the MR signal (that is, the duration of operation of the analog-to-digital converter, ADC 512) divided by the duration of the entire sequence module, becomes lower for higher sampling bandwidth. To improve the efficiency of the tracking sequence module 500, the tracking pulse sequence module 500 of FIG. 5A may be modified to create a modified tracking pulse sequence module 500′ as illustrated in FIG. 5B.

As illustrated in FIG. 5B, instead of using a separate RF excitation for each projection, one non-selective excitation pulse 502′ may be used for the acquisition of all three projections. Due to the small flip angle needed for the active marker tracking, the hard RF pulse can be shortened to the order of 100 μs. In this work, 60 μs was used.

The pre-positioning 504′ and readout gradients 506′ are also used as spoiler gradients. This is similar to the tracking pulse sequence 500 of FIG. 5A, except that instead of having a spoiler gradient 510 in one perpendicular axis, the purpose of the spoiler gradients is achieved by applying the pre-positioning gradient 504′ in both the other two axes at the same moment as the spoiler gradients 510 were applied in FIG. 5A. By doing so, no pre-positioning gradient is needed between the acquisitions of the X, Y and Z projections, as shown in FIG. 5B when compared with FIG. 5A. A dotted line is used to show the timing of the ADC 512′ and gradients 506′. Thus, as can be seen, the gaps between the ADC 512′ periods are limited only by the ramp-up and ramp-down times of the readout gradients 506′. Thus, the pre-positioning gradients 504′, as well as the rewinding gradients 508′, for all three axes are simultaneously performed, and the gradient waveforms in all axes are highly overlapped.

Referring to FIG. 5C, the modifications to the k-space sampling trajectory resulting from the modified tracking pulse sequence module 500′ (dashed lines to dot-dash lines 550) are illustrated. The origin of the k-space 552 is at the center of the cube 554.

Thus, the tracking sequence module 500 of FIG. 5A provides a fast and accurate method to monitor subject motion. Marker locations can be measured in 13 to 50 ms, allowing a location update frequency up to 77 frames-per-second (FPS). Although 13 ms duration of the tracking sequence module 500 is already short, a faster module is still desirable. First, a faster tracking sequence module requires less overhead added to the host MR sequence for marker-based motion correction in MRI or MR-PET acquisitions. Second, a fast tracking sequence can provide more accurate localization by reducing the time difference between the measurements of the projections (such as the X, Y and Z projections). Last but not the least, the fast tracking sequence enables the monitoring of rapid motion with high update frequency.

Thus, the tracking sequence module 500′ of FIG. 5B provides an efficient k-space trajectory for active marker tracking that measures all three X, Y and Z projections with one short RF excitation in a time shorter than the tracking sequence module 500. The tracking sequence module 500′ can obtain the locations of the markers within 5 ms which is equivalent to 200 FPS.

Hence, as described, present disclosure provides a pulse sequence that does not rely on “navigators” or a “navigator sequence” that rapidly acquires a crude low spatial resolution version of the MR image, for example a simple 1D projection of the subject, and or methods that use such navigator data to either correct the incoming raw data as it is acquired, or to adjust the scanner operation (such as shifting the image plane), or else is saved for motion correction in post processing. Rather, as described, MR data is acquired from a set of tracking coils. To this end, the above-described methods can be implemented even if MR imaging (including navigator imaging) of the subject is not being conducted, and even if the MR apparatus (transmit and receive coils) that are required for MR imaging are absent from the MRI scanner. Further, the systems and method of the present disclosure do not require or use of image data from the subject and, as described, can optionally use data acquired from a sample that is separate from the subject.

To demonstrate the utility of the proposed efficient trajectory, a phantom study was performed in the context of MR active marker based motion correction for PET imaging in simultaneous MR-PET acquisition. The acquisitions were performed on a simultaneous MR-PET scanner (Siemens Biograph mMR, Siemens Healthcare, Erlangen, Germany).

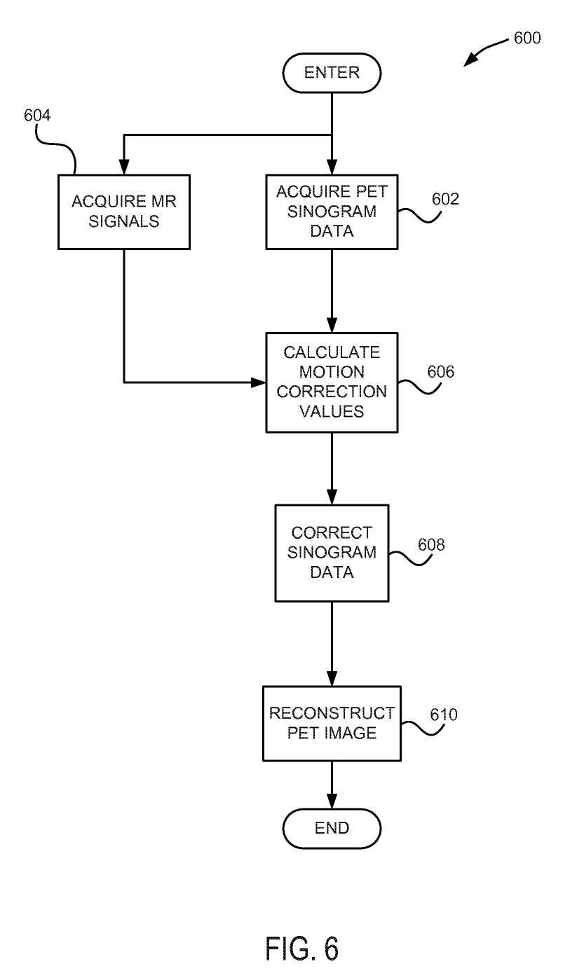

Referring to FIG. 6, steps of one motion correction process 600 are illustrated. At process block 602, sinogram data is acquired by the PET system, while MR or NMR signals are also acquired using the MRI system at step 604. As illustrated, process blocks 602 and 604 occur substantially contemporaneously. Next, motion correction values are calculated at process block 606 from the MR signals acquired in process block 604. The motion correction values are then used to correct the acquired sinogram data at process block 608. In this process, both the MR signals and sinogram data receive a time stamp when they are acquired such that a MR signal that is acquired at substantially the same instance in time as a given sinogram is employed to correct for subject motion at that moment in time. After the sinogram data has been corrected for subject motion, PET images are reconstructed at process block 610 that are corrected for any motion.

More particularly, motion corrected PET image reconstruction will now be described. Several methods have been proposed for PET motion correction. One method is to divide detected events into multiple static motion phases using a specified threshold and perform individual reconstruction in each phase. This is then followed by registering each frame to a reference motion phase and summing all the registered images. However, a high threshold can cause the motion within the phase to be ignored. On the other hand, a low threshold can lead to low statistical phase to be reconstructed. Lack of an adequate number of acquired events in the individual phases can, in turn, adversely affect the quality of the final reconstructed images. Moreover, an increased number motion phases can lead to increased computation time. Another approach is to perform deconvolution on the motion-blurred reconstructed images. However, the deconvolution amplifies the noise in the PET data. When the motion is significant, it also requires spatially variant deconvolution filters, which increase computational costs and introduce other artifacts. Another method is to model the motion in the PET system matrix in PET reconstruction. This method is usually applied to non-rigid motion. Although such a method can also be applied to rigid motion, it would converge slowly. For rigid motion, such as head motion, the most accurate approach is to correct the individual lines of response.

Most regions of interest studied in PET, such as the head, undergoes rigid body motion. Thus, the motion of the object can be uniquely determined by the locations of multiple non-collinear active markers, such as using the above-described tracking coil systems illustrated in FIG. 4. To do so, the transformation matrices from a given location to a pre-determined reference location can be calculated. Then, the motion can be incorporated into a list-mode ordinary Poisson expectation maximization algorithm as follows:

$${x}_{j}^{k + 1} = \frac{{x}_{j}^{k}}{{\overline{s}}_{j}} {\sum }_{n = 1}^{N} \frac{{a}_{{i}_{n} , j}^{m}}{{\sum }_{{j}^{′}}^{J} {a}_{{i}_{n} , j}^{m} {x}_{{j}^{′}}^{k} + \overline{S} {c}_{n} + {\overline{R}}_{n}} ; \qquad ( 1 )$$

where xⱼ^{k} is the activity in voxel i (i=1,2, . . . ,J) at the k^{th} iteration, s̅ⱼ is the average sensitivity for voxel j over all motion phases where m denotes the motion phase number, iₙ (iₙ=1,2, . . . , I) denotes the possible LORs of the PET scanner, aᵢ_{ⁿ}ⱼ^{m} is an element of the system matrix in which the measured motion is incorporated (the probability for an event in voxel j at motion phase m to be detected along LOR iₙ; S̅c̅ₙ and R̅ₙ are the average scatter and random along LOR iₙ.

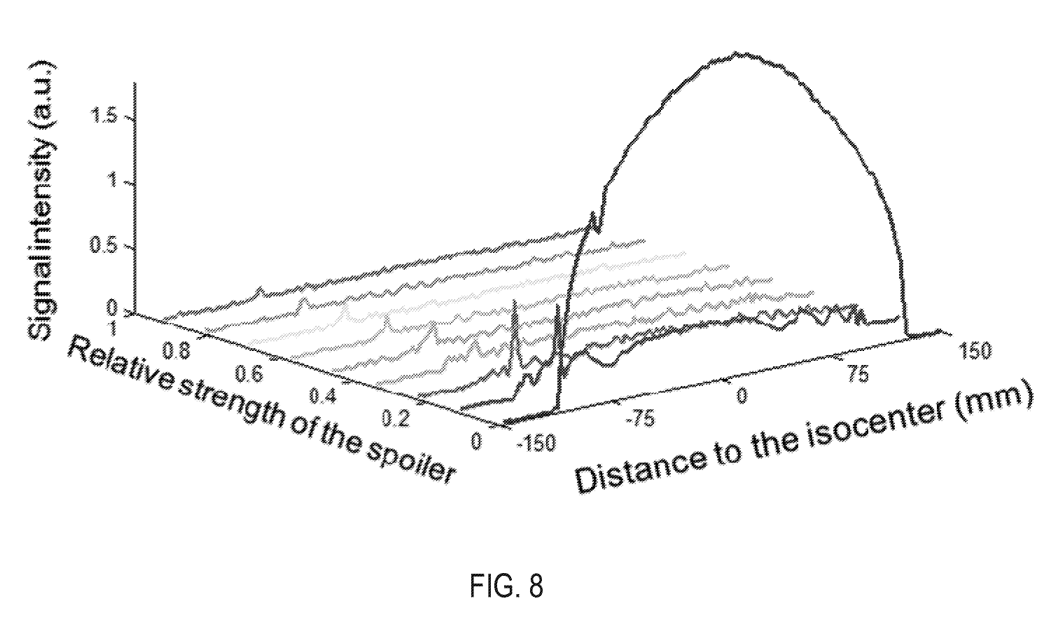

In particular, FIG. 8 shows the X projections of one tracking coil attached to a cylindrical water phantom acquired with varying spoiler strength using the tracking sequence module of FIG. 5A, where the relative strength of the spoiler is defined as the ratio of the area of the spoiler to that of the pre-positioning gradient. It can be seen that the spoiler indeed preserves signal from the active marker while eliminate the signal from large volume. Although the spike of the active marker was the highest when the relative strength of the spoiler was approximately 0.2, the background signal wasn't fully depleted at that strength. The background signal was eliminated when the relative strength increased to 1, while the spike of the marker was still preserved.

Testing results show that the efficient trajectory for active marker tracking of the present disclosure provides fast and accurate marker location measurements. Using the efficient trajectory, the duration of the active marker tracking sequence module has been shortened to 5.5 ms. Sub-5ms duration of the tracking module can be achieved with slightly varied parameters: pixel size=1.52 mm, 256 samples per line, 2 times readout oversampling, bandwidth=1502 Hz/pixel, allowing an update rate higher than 200 FPS; whereas, to our knowledge, the reported shortest duration of the conventional tracking sequence module in the literature is 12.9 ms10.

In one set of experiments, the body RF coil was used to wirelessly receive the signal emitted by the active markers. In practice, bird-cage or multichannel head coils can be used to boost the SNR of the signals from the markers. For multichannel coils, combination of data from the channels can be performed by a simple sum-of-squares.

Certain errors may occur during position tracking with coils or microcoils. For example, due to the presence of copper wires and glass in the vicinity of the doped water samples in the active markers, a local magnetic susceptibility induced field shift is expected. The water sample might not be perfectly spherical because the water might enter the neck of the cell, or a bubble caused by insufficient filling may be present; this effect will create a small susceptibility shift, as well as possible broadening and asymmetry of the profile. Both of these sources of shift will exhibit a dependence on orientation of the marker.

The present disclosure studied how the susceptibility affects the accuracy of the location measurements. This effect can be measured by repeating the tracking module with opposite gradient polarity. This study was performed on one wireless active marker with the same acquisition parameters as used in the motion correction study except the readout bandwidths were varied (130, 201, 300, 592, 814 and 1149 Hz/pixel). The readout bandwidth is proportional to the magnitude of the readout gradient. A stronger readout gradient yields a more accurate position because it dominates small field shifts caused by other effects such as magnetic susceptibility, but reduces the SNR.

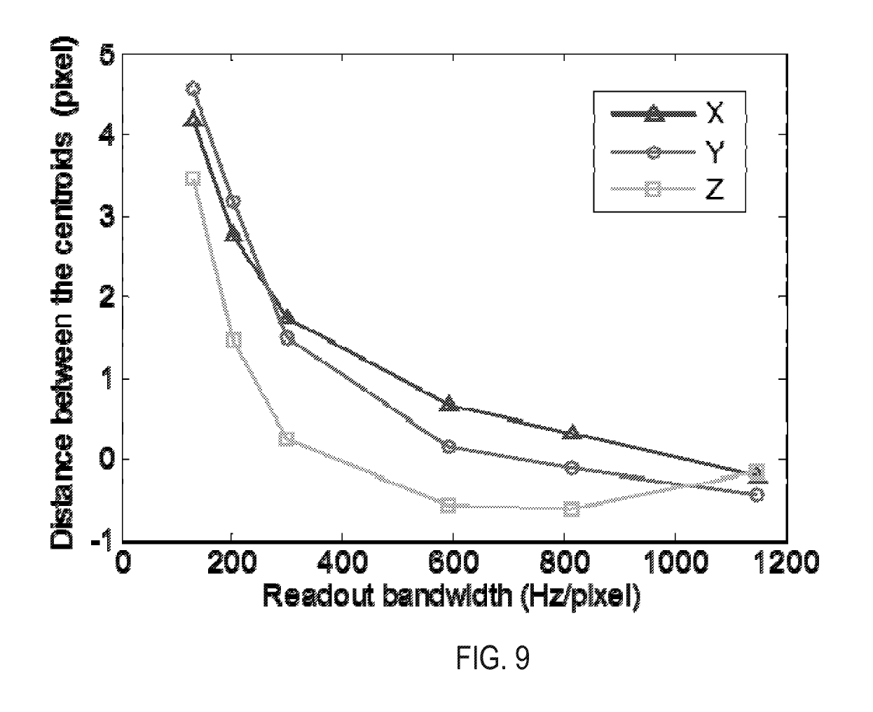

The difference between the centroids of the X, Y and Z projections read out with gradients of opposite polarity were calculated for each readout bandwidth and the results are shown in FIG. 9. The susceptibility induced marker peak shift is not negligible for low bandwidths. When the bandwidth was 130 Hz, the distance discrepancies between the centroids in the X, Y and Z projections were 4.2, 4.6, 3.5 pixels, respectively. The distances decrease as the bandwidth increases. When the bandwidth was 1149 Hz/pixel, the distance discrepancies were 0.21, 0.44, 0.15 pixels for X, Y and Z projections. Note that the susceptibility induced peak shift is half of the distance shown here. This means the location measurement error caused by susceptibility is less than 0.22 pixel for a bandwidth of 1149 Hz/pixel, which is equivalent to 0.26 mm with the acquisition parameters used. This error is negligible for most applications. However, when lower bandwidth is needed, especially when the bandwidth is lower than 600 Hz/pixel, the susceptibility induced location measurement error can no longer be ignored. This can be addressed by either applying a periodically measured correction, or by performing the proposed sequence module with two polarities and using the average location.

When lower bandwidth is used, the efficiency improvement of the proposed trajectory will be less. The efficiency improvement is largest for high bandwidth since the proposed trajectory improves the efficiency by reducing time spent on magnetization preparation. The magnetization preparation time will not change much when the bandwidth is reduced, but the ADC duration will be longer. However, in practice, the highest achievable bandwidth is almost always desired because the sampling time needed for the tracking module increases with the decrease of bandwidth.

Although it is intuitive to assume perfect spatial alignment between PET and MR, the spatial accuracy of MR is affected by gradient non-linearity induced spatial distortion especially at the locations far from the scanner isocenter. When not corrected, the spatial distortion can lead to misalignment between the attenuation map and emission image, and error in motion tracking. The gradient non-linearity was measured when the scanner was installed and the parameters are stored on the scanner, allowing one to account for it. The spatial distortion correction can be performed off-line (Jovicich et al., 2006). The spatial distortion correction was not performed in these studies because the markers were close to the isocenter of the scanner, where the distortion caused by gradient non-linearity was small. Also, due to the small displacements of the markers, gradient non-linearity had only second-order effects on the accuracy of our measurements.

The MR tracking sequence module shown in FIGS. 5A and 5B can be incorporated into other clinical MR imaging sequences. Because of the small flip angle needed (for example, 1 degree), minimal impact to the host MR sequence is ensured. As a result, this tracking module can be incorporated into most gradient-echo (including ultra-short TE), spin-echo, and echo-planar imaging sequences with the exception of certain steady-state free precession sequences. Also, by incorporating the module into other MR sequences, it allows motion corrected MR acquisition. The same motion measurements can be used to perform prospective motion correction during MR acquisition. This can fully exploit the potential of simultaneous PET-MR scans in brain imaging. Besides brain PET and MR imaging, the proposed technique could also aid acquisitions in areas where subject bulk motion affects the results such as dynamic cardiac perfusion studies.

In some tests, the traces from the tracking coils do not overlap. In practice, the traces may cross each other if the markers are not placed far apart from each other. When that happens, the correspondence between the markers and the traces can still be determined by utilizing the constraint that the distances between the active markers remain the same under rigid motion. One measurement generates three locations on each of the X, Y and Z projections. In total, there are 3³=27 possible 3D locations for the three markers. Of these, there are 36 possible combinations forming a triangle, as they must. The triangle with the edge lengths that match with the initial placement will be selected. Moreover, if the locations were frequently measured (as was done in this work), the constraint that the displacement of each marker is small between two measurements can also be used for the correspondence determination.

The motion tracking and correction with the proposed efficient trajectory was demonstrated in retrospective motion corrected PET image reconstruction. However, the techniques described herein can also be used in prospective motion correction in MR imaging or catheter tracking. The improved acquisition efficiency can reduce the overhead for motion tracking or improve the temporal resolution.

Thus, a system and method for efficient active marker tracking is provided for correcting PET data using MR data in a combined or simultaneous MR-PET scanner. The tracking sequence module based on the proposed trajectory can obtain the locations of multiple active markers within 5 ms and is significantly more efficient than conventional tracking sequence modules. The improved efficiency of the tracking sequence module leads to less overhead for motion tracking and potentially higher frame rates for rapid motion.

It should be apparent that many variations from the above embodiments are possible without departing from the invention. For example, the MRI system and PET scanner may be more fully integrated with control and processing components being shared by both systems. As another example, the material detected by the tracking coils may be contained with the coils or may be adjacent to the exterior of the coils. As another example, the tracking coils may be wired to the MRI scanner or may operate wirelessly via inductive coupling to the body RF coil or another coil in the scanner. As still another example, wired tracking coils may be used in transmit/receive mode, whereby the RF excitation is provided through the wired connection rather than via the body RF coil. As yet another example, the tracking coils can have winding configurations other than solenoids.

1. A medical imaging system comprising:
a positron emission tomography (PET) system for acquiring a series of medical images of a subject, the PET system comprising:
a plurality of detectors arranged about a bore configured to receive the subject and to acquire gamma rays emitted from the subject as a result of a radiotracer administered to the subject and configured to communicate PET signals corresponding to acquired gamma rays;
a magnetic resonance imaging (MRI) system for acquiring a series of medical images of the subject from within the bore, the MRI system comprising:
a magnet system configured to generate a polarizing magnetic field about at least a portion of the subject arranged in the bore;
a plurality of gradient coils configured to apply a gradient field to the polarizing magnetic field;
a radio frequency (RF) system having a plurality of motion tracking coils positioned, the RF system configured to acquire MR data from the motion tracking coils, wherein each motion tracking coil includes a conductor extending through a plurality of loops to form a coil;
an MRI computer system programmed to control the RF system to receive MR data from the RF system;
a data processing system configured to:
receive the MR data and determine motion of the subject from the MR data by determining a position of the motion tracking coils over time; and
receive the PET signals and correct the PET signals using the motion of the subject determined from the MR data to generate corrected PET images of the subject.

2. The medical imaging system of claim 1 further comprising a sample material separate from the subject and wherein the motion tracking coil is configured to acquire MR data from the material sample.

3. The medical imaging system of claim 1 wherein the sample material comprises a water sample or other samples capable of emitting MR detectable signal arranged within a housing extending through the interior.

4. The medical imaging system of claim 1 wherein the plurality of motion tracking coil includes at least three motion tracking coils distributed in a non-collinear arrangement.

5. The medical imaging system of claim 1 wherein the motion tracking coils communicate with the medical imaging system by magnetic inductive coupling with at least one RF receive coil of the MRI system.

6. The medical imaging system of claim 1 wherein the MR computer system is configured to control the RF system to perform a pulse sequence that includes a single RF excitation pulse followed by a plurality of gradient pulses to acquire MR data from the plurality of motion tracking coils along three directions.

7. The medical imaging system of claim 6 wherein the plurality of gradient pulses includes a plurality of pre-prepositioning gradient pulses followed by respective readout gradients and wherein the pre-positioning gradient pulses are applied in both two axes at the same time to remove signal from subject from being received by the motion tracking coils.

8. The medical imaging system of claim 1 further comprising a wired connection between the plurality of motion tracking coils and the MRI system to communicate the MR data

9. The medical imaging system of claim 1 wherein the data processing system is configured to perform a list-mode reconstruction to generate corrected PET images of the subject.

10. The medical imaging system of claim 9 wherein the data processing system is configured to determine transformation matrices from a given location in a field of view of the medical imaging system to a pre-determined reference location in the field of view using the MR data and incorporate the transformation matrices into a list-mode ordinary Poisson expectation maximization algorithm to generate the corrected PET images of the subject.

11. A method for generating a positron emission tomography (PET) image corrected for subject motion in a combination PET and magnetic resonance imaging (MRI) system, the method including steps comprising:
a) preparing a subject to be imaged in a combined PET and MR imaging process by administering a radionuclide to the subject and arranging a plurality of motion tracking coils about the subject, wherein each of the plurality of motion tracking coil includes a conductor extending through a plurality of loops to form a coil configured to receive signals from a sample material;
b) acquiring, with a PET imaging system, sinogram data that counts a number of coincidence events at a plurality of lines of response (LOR);
c) periodically acquiring, with the MRI system, over a time period that extends concurrently with step b), a plurality of MR signals generated using the plurality of motion tracking coils arranged about the subject;
d) calculating, from the MR data, motion data; and
e) correcting the sinogram data using the motion data to generate corrected PET images of the subject.

12. The method of claim 11 where step c) includes performing a pulse sequence that includes a single non-selective RF excitation pulse followed by a plurality of gradient pulses to acquire MR data from the plurality of motion tracking coils along three orthogonal directions.

13. The method of claim 12 wherein the plurality of gradient pulses includes a plurality of pre-prepositioning gradient pulses followed by respective readout gradients and wherein the pre-positioning gradient pulses are applied in both two axes at the same time to remove signal from subject from being received by the motion tracking coils.

14. The method of claim 11 wherein step e) includes performing a list-mode reconstruction to generate corrected PET images of the subject.

15. The method of claim 14 wherein step e) includes determining transformation matrices from a given location in the subject to a pre-determined reference location in the subject using the MR data and incorporating the transformation matrices into a list-mode ordinary Poisson expectation maximization algorithm to generate the corrected PET images of the subject.

16. The method of claim 11 wherein step a) includes distributing the plurality of motion tracking coil about the subject in a non-collinear arrangement.

17. The method of claim 11 wherein the sample material includes at least one of a water sample and a portion of the subject.

18. The method of claim 11 further comprising causing the motion tracking coils to communicate with the MRI system by magnetic inductive coupling of the motion tracking coils with at least one RF receive coil of the MRI system.
